(19) United States
(12) Patent Application Publication
(10) Pub. No.: US 2017/0086736 A1
(43) Pub. Date: Mar. 30, 2017

(54) METHODS AND SYSTEMS USING NON-LABELED ANTIMETABOLITES AND ANALOGS THEREOF AS THERANOSTIC AGENTS

(71) Applicant: The Johns Hopkins University, Baltimore, MD (US)

(72) Inventors: Guanshu Liu, Timomium, MD (US); Yuguo Li, Baltimore, MD (US); Peter C.M. Van Zijl, Ellicot City, MD (US); Shinbin Zhou, Owings Mills, MD (US); Bert Vogelstein, Baltimore, MD (US)

(73) Assignee: The Johns Hopkins University, Baltimore, MD (US)

(21) Appl. No.: 15/123,942

(22) PCT Filed: Mar. 6, 2015
(86) PCT No.: PCT/US15/19291

§ 371 Date: Sep. 6, 2016

Related U.S. Application Data
(60) Provisional application No. 61/949,044, filed on Mar. 6, 2014.

(51) Int. Cl. A61B 5/00; G01R 33/56; A61B 5/055

(52) U.S. Cl. CPC A61B 5/4848; A61B 5/055; G01R 33/5608

(57) ABSTRACT

A method, computer-readable medium and system of planning, guiding and/or monitoring a therapeutic procedure, can include: receiving a non-labeled therapeutic agent by a subject, said non-labeled therapeutic agent comprises at least one type of water-exchangeable proton that is exchangeable with protons in surrounding water molecules so as to enhance detection by a chemical exchange saturation transfer (CEST) process; acquiring a plurality of CEST magnetic resonance images of said non-labeled therapeutic agent within a region of interest of said subject for a corresponding plurality of times; and assessing at least one of a therapeutic plan or therapeutic effect of said non-labeled therapeutic agent in tissue of said subject based on said plurality of magnetic resonance images

### CROSS REFERENCE TO RELATED APPLICATIONS

This application claims priority to U.S. Provisional Patent Application No. 61/949,044, filed Mar. 6, 2014, which is hereby incorporated herein by reference in its entirety.

### FEDERAL FUNDING

This invention was made with Government support of Grant No. R21EB015609 awarded by the Department of Health and Human Services, The National Institutes of Health (NIH). The U.S. Government has certain rights in this invention.

### TECHNICAL FIELD

This disclosure relates generally to assessing non-labeled therapeutic agents as theranostic agents and more particularly to methods, systems and media for assessing the non-labeled therapeutic agents.

### BACKGROUND

1. Field of Invention

The field of the currently claimed embodiments of this invention relates to systems and methods of using non-labeled antimetabolites and analogs thereof as theranostic agents.

2. Discussion of Related Art

Imaging drug delivery is of great clinical importance. Achieving effective anticancer drug therapy requires not only the effectiveness of an anticancer drug to act against a particular type of cancer cells, but also the delivery of the drug so as to exceed a threshold effective level of drug activity in the full anatomic extent of the cancer cell population. For instance, the heterogeneity in cancer architecture, especially in the vasculature anatomy and the related tissue barrier functions, also determines the success of the administered drug^{3,4}, which are often unpredictable in an individual patient. It is essential to develop tools to assess whether drugs are delivered to the tumor at an adequate concentration in each individual patient and subsequently adjust the treatment plan accordingly, a so-called “personalized medicine” strategy⁵, in which non-invasive imaging modalities are believed to play a central role. Currently there is an extensive investment in the development of molecular imaging techniques that can stratify patients to select appropriate patients for treatment and to provide early proof of response⁶. As a result, for these drugs to advance to the clinic, the Food and Drug Administration (FDA) has provided its vision of the future in the form of the Critical Path Initiative, where molecular imaging will play a pivotal role in hastening drug development in early clinical trials and will also provide data critical for subsequent approval.

However, most currently available molecular imaging modalities rely heavily on the use of imaging tags, e.g., radioactive compounds for PET/SPECT and metallic compounds for MRI. In MRI, metallic agents are widely used to track the delivery of drug carriers such as Mn²⁺-based⁷ and Gd³⁺-based⁸ T1 agents and iron-based T2* agents⁹. Several challenges arise from the use of extra imaging tags. First, MRI detection relies on the signal of imaging agents, which do not necessarily reflect correct information (e.g. concentration and location) of the drug unless they are conjugated together. Second, the incorporation of extra imaging tags into the drug or drug delivery systems could potentially change the physico-chemical properties and affect the delivery. Moreover, there are various regulatory, financial, and practical barriers that prevent the imaging agents that have been developed pre-clinically from being translated quickly to the clinic to play an important role in accelerating clinical trials of new therapeutics. All these led us to explore a totally new “label-free” approach.

While some label-free imaging techniques have been developed directly based on the inherent signal of drugs, for example, the inherent fluorescence signal of doxorubicin¹⁰ or the inherent fluorine NMR signal of 5-fluorouracil (5-FU)¹¹, an approach suitable for a broad array of drugs is still lacking Therefore, there remains a need for improved systems and methods using label-free imaging techniques.

### SUMMARY

A method of planning, guiding and/or monitoring a therapeutic procedure can include: receiving a non-labeled therapeutic agent by a subject, the non-labeled therapeutic agent comprising at least one type of water-exchangeable proton that is exchangeable with protons in surrounding water molecules so as to enhance detection by a chemical exchange saturation transfer (CEST) process; acquiring a plurality of CEST magnetic resonance images of the non-labeled therapeutic agent within a region of interest of the subject for a corresponding plurality of times; and assessing at least one of a therapeutic plan or therapeutic effect of the non-labeled therapeutic agent in tissue of the subject based on the plurality of magnetic resonance images.

A non-transitory, computer-readable storage medium for planning, guiding and/or monitoring a therapeutic procedure can include computer-executable instructions that, when executed by a computer, cause the computer to perform: acquiring a plurality of chemical exchange saturation transfer (CEST) magnetic resonance images of a non-labeled therapeutic agent that has been received by a subject, wherein the acquiring step acquires the plurality of magnetic resonance images within a region of interest of the subject for a corresponding plurality of times, the non-labeled therapeutic agent comprising at least one type of water-exchangeable proton that is exchangeable with protons in surrounding water molecules so as to enhance detection by a CEST process; and assessing at least one of a therapeutic plan or therapeutic effect of the non-labeled therapeutic agent in tissue of the subject based on the plurality of magnetic resonance images.

A system for planning, guiding and/or monitoring a therapeutic procedure can include: a data processing system; and a display system configured to communicate with the data processing system, where the data processing system comprises non-transitory, computer-executable instructions that, when executed by the data processing system, causes the data processing system to perform: acquiring a plurality of chemical exchange saturation transfer (CEST) magnetic resonance images of a non-labeled therapeutic agent that has been received by a subject, wherein the acquiring step acquires the plurality of magnetic resonance images within a region of interest of the subject for a corresponding plurality of times, the non-labeled therapeutic agent comprising at least one type of water-exchangeable proton that is exchangeable with protons in surrounding water molecules so as to enhance detection by a CEST process; and assessing at least one of a therapeutic plan or therapeutic effect of the non-labeled therapeutic agent in tissue of the subject based on the plurality of magnetic resonance images.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

FIG. 1 shows an illustration of Chemical Exchange Saturation Transfer (CEST) and drugCEST, according to an embodiment of the present invention.

FIG. 2 shows an illustration of the potential roles of drugCEST MRI can exert in preclinical research and clinical practice, according to an embodiment of the present invention.

FIG. 3 shows a comparison of the detectability of CEST MRI and 19F MRSI, according to an embodiment of the present invention.

FIG. 4 shows a demonstration of using the pH dependency of multiple drugCEST signal carried by the same drug to assess the environment pH and consequently the location of liposomes, according to an embodiment of the present invention.

FIG. 5 shows a schematic of the proposed self-trackable multifunctional liposome system using the CEST signal of gemcitabin, according to an embodiment of the present invention.

FIG. 6 illustrates in vivo detection of gemcitabine using the proposed CEST MRI technology in a Capan-1 human pancreatic cancer xenograft, according to an embodiment of the invention.

FIG. 7 illustrates the dynamic CEST MRI study of the tumor uptake of received gemcitabine in a mouse bearing Panc 253 patient-derived tumors, according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The phrase “non-labeled therapeutic agent” refers to a solution, a dispersion, a powder, a tablet or any other administrable form of composition that comprises molecules that are not radioactive, not paramagnetic, and do not contain non-abundant magnetically enriched isotopes. It can be, or can include, a drug in any administrable form, including, but not limited to, drugs in delivery vehicles, such as nanoparticles. It can include anti-cancer drugs, a drug analog and/or a drug modulator.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® NT/98/2000/XP/Vista/Windows 7/8/etc. available from MICROSOFT® Corporation of Redmond, Wash., U.S.A. or an Apple computer executing MAC® OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or other camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

Some embodiments of the current invention are directed to the use of the MRI signal carried on the drug molecules for non-invasively detecting and quantifying the administered drugs using chemical exchange saturation transfer (CEST) MRI. Our approach allows transforming of currently available drugs, drug analogs and drug delivery systems, including those already in the clinic and those still under pre-clinical development, to be theranostic agents, without any radioactive-, paramagnetic- or super-paramagnetic-based labeling. This approach can allow the MRI monitoring of the drug delivery, assessment of the drug resistance, predicting of drug penetration to tumor stroma, and stratification of patients in clinical trials and clinical practices. This technology may be used as, but is not limited to, a clinical imaging package for stratifying patient before and/or during chemotherapy to select patients with the appropriate treatment plan.

Some embodiments are directed to the use of the MRI signal carried on the molecules of antimetabolites for non-invasively detecting and quantifying the administered anticancer drugs using chemical exchange saturation transfer (CEST) MRI. An approach can allow transforming of three categories of currently available metabolites (purine-, pyrimidine- and folate-based) and their analogs, as well as drug delivery systems containing these agents (including those already in the clinic and those still under pre-clinical development) to be theranostic agents, without any radioactive-, paramagnetic- or super-paramagnetic-based labeling. This approach can allow the MRI monitoring of the drug delivery, assessment of the drug resistance, predicting of drug penetration to tumor stroma, and stratification of patients in clinical trials and clinical practices. This technology can be used as, but is not limited to, a clinical imaging package for stratifying patient before and/or during chemotherapy to select patients with the appropriate treatment plan.

Papers related to background and conventional methodologies are provided below. MRI pulse sequences that can be used for the data acquisition, as the chemical exchange saturation transfer (CEST) technologies have been patented before (see below B1-B3). Some embodiments of the current invention are directed to the new clinical indication of drugs and drug analogs as theranostic agents, and the MRI detection together with extents for processing and displaying the data.

B1: Balaban; Robert S. (Bethesda, Md.), Ward; Kathleen M. (Arlington, Va.), Aletras; Anthony H. (Rockville, Md.); U.S. Pat. No. 6,963,769; PCT/US00/10878, published Nov. 8, 2005.

B2: van Zijl, Peter (Ellicott City, Md.), Jones, Craig (Ilderton, Canada), U.S. Pat. No. 7,683,617; PCT/US2006/028314, Mar. 23, 2010.

B3: van Zijl, Peter (Ellicott City); Kim, Mina and Gillen, Joseph. Frequency Referencing Method for Chemical Exchange Saturation Transfer (CEST) MRI; JHU disclosure C10151, 2007.

A specific MRI technology that can be used according to some embodiments to accomplish a goal is called Chemical Exchange Saturation Transfer (CEST)¹². According to an embodiment, a method of planning, guiding and/or monitoring a therapeutic procedure is disclosed. While various embodiments of this method are disclosed as a method throughout this section, it is to be understood that a non-transitory, computer readable medium or a data processing system can include instructions that when executed by at least one computer or data processing system cause a computer or data processing system to perform analogous steps to the method embodiment. One embodiment can include receiving a non-labeled therapeutic agent by a subject. The term “receive” is intended to be broadly defined to encompass dispersing, administering, dispensing, applying, delivering, distributing, infusing and/or supplying the therapeutic agent into the subject. The non-labeled therapeutic agent can include at least one type of water-exchangeable proton that is exchangeable with protons in surrounding water molecules so as to enhance detection by a chemical exchange saturation transfer (CEST) process. The non-labeled therapeutic agent can be at least one of a drug, a drug analog, or a drug modulator. The non-labeled therapeutic agent can be an anticancer drug. The non-labeled therapeutic agent can be a drug delivery system. In this embodiment, the drug delivery system can be a nanoparticle drug delivery system. Thus, a subject can receive a non-labeled therapeutic agent.

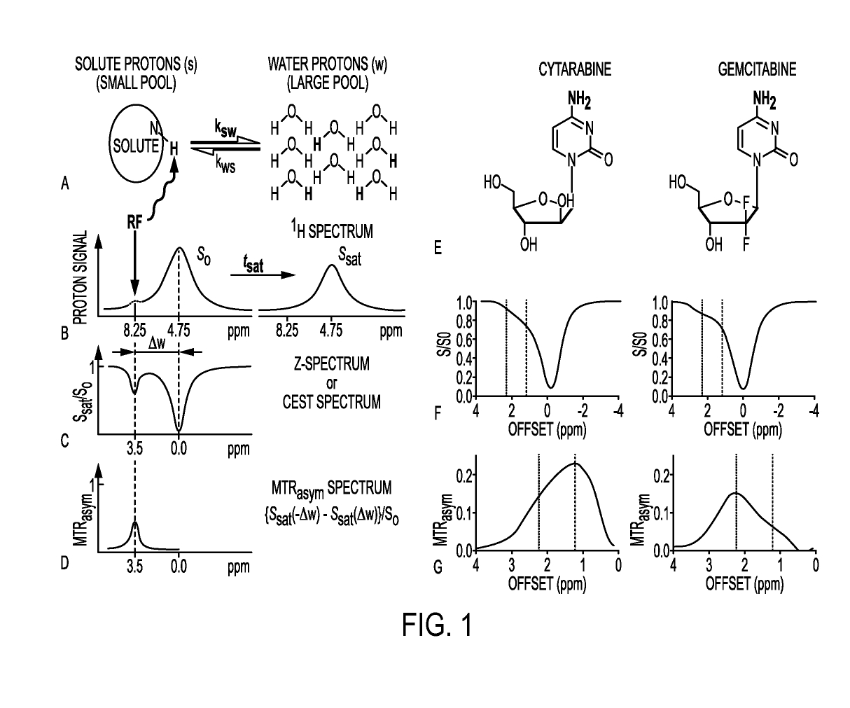

As illustrated in FIG. 1, the presence of exchangeable protons, referring to labile protons that physically jump from one molecule (i.e., the solute or CEST agent resonant at a specific offset, i.e., Δω ppm) to another (i.e., water, assigned to 0 ppm), allows the transfer of NMR signal from a solute to its surrounding water molecules, resulting in a change in MRI signal (the NMR signal of water), for example, as a decreased signal due to the transferred saturation (the attenuated NMR signal). This would of course correspond only to an mM or μM change for the drug and would not be detectable on the large MRI signal of 55 M water molecules (110 M protons). However, for every saturated proton leaving from the solute to water, an intact proton relocates from water to the solute, a process so-called chemical or proton exchange, and subsequently gets saturated again, which will be transferred to the water again in the next exchange. If the exchange rate is fast enough, this continuous process can lead to great signal amplification of the NMR signal carried by the agent, even up to thousands of times (depending on the exchange rate). This can then cause a several percentage decrease in the water signal, allowing imaging of the low-concentration agent with the sensitivity of the water signal. The percentage of water signal decrease, or CEST contrast, is traditionally described by magnetization transfer ratio asymmetry (MTR_{asym}) defined by MTR_{asym}=(S^{-Δω}−S^{+Δω})/S₀, where S^{-Δω} and S^{-Δω} are the MRI signal with RF irradiation at particular offsets +Δω and −Δω respectively, and So is that acquired without RF saturation (element (d) of FIG. 1). Because having detectable exchangeable protons (at an appropriate rate at an adequate concentration) is a requirement for generating CEST MRI signal, it thus opens the possibility to detect natural biocompatible molecules by their CEST signal, potentially improving the clinical translatability.

One embodiment can include acquiring a plurality of CEST magnetic resonance images of the non-labeled therapeutic agent within a region of interest of the subject for a corresponding plurality of times. In an embodiment, computer readable media can include instructions that, when executed, cause a computer to perform acquiring a plurality of CEST magnetic resonance images of a non-labeled therapeutic agent that has been received by a subject. In this embodiment, the acquiring step can acquire the plurality of magnetic resonance images within a region of interest of the subject for a corresponding plurality of times. The non-labeled therapeutic agent can include at least one type of water-exchangeable proton that is exchangeable with protons in surrounding water molecules so as to enhance detection by a CEST process.

As is shown in FIG. 1, two widely used cytidine-analog-based anti-tumor drugs, Cytarabine (araC) and Gemcitabine (Gem), also have a strong CEST signal, very similar to that of cytosine, indicating that the rational discovery of drugs with CEST signal is possible based on the similarity in chemical structure. Therefore, this can be expanded to other antitumor categories and the biochemical drug modulators (a non-toxic agent that can enhance the effect of drugs when used with specific drugs, but has no effect when used alone), on which we demonstrate how to transform currently available drugs, including those already in the clinic and those still under pre-clinical development, to be MRI-detectable, without any radioactive-, paramagnetic- or super-paramagnetic-based labeling. We refer to the CEST of drugs and drug modulators collectively as drugCEST.

Elements (a)-(d) of FIG. 1 show an illustration of CEST contrast (taken from reference [¹] with permission). Element (a) shows RF-saturated protons in the small solute (s) pool exchange with protons in the bulk water (w) pool, resulting in small undetectable change of MRI signal. However, this process is repeated continuously, leading to effect amplification of magnitude depending on the exchange rate (k_{sw}). The CEST contrast can be quantified by a NMR spectrum element (b), a z spectrum in element (c) of frequency dependence of normalized saturated water signal Sₛₐₜ/S0, with S0 the non-saturated signal) or, an MTRasym plot in element (d). Elements (e)-(f) show CEST detection of drugs through their carried exchangeable protons on NH₂ (blue) and OH (red) as exemplified by araC (left) and Gem (right) in PBS solution (20 mM, pH 7.4) at 37° C. using a standard CEST MRI method (RF pulse field strength (B1)=3.6 μT and duration =3 s). Element (e) shows the chemical structures of the two agents. Elements (f)-(g) of FIG. 1 show the CEST contrast of them as quantified by z-spectra and MTR_{asym} plots respectively, showing strong CEST contrast at ˜1 and ˜2 ppm corresponding to OH and NH₂ respectively, which appear partially overlapped at pH 7.4 but can be resolved at low saturation power.

Our approach is distinctive from most conventional molecular imaging techniques because it is “label-free” and directly exploits the signal originating from the drugs themselves. Unlike techniques that rely on the use of imaging agents, our approach can directly detect the effective dose of the administered drugs in each sub-region of tumors, providing direct information of the spatial distribution and temporal dynamic of the drugs, enabling personalized chemotherapy. More importantly, if drugs are detected without extra labeling, we will be able to directly transform currently available drugs into “imageable drugs” and the clinical translation of them will have minimal if any barriers. Consequently, our approach can promote a shift of the clinical evaluation of drug effectiveness from delayed endpoints (often months) to early time points (hours and days).

One embodiment can also include assessing at least one of a therapeutic plan or therapeutic effect of the non-labeled therapeutic agent in tissue of the subject based on the plurality of magnetic resonance images. For example, in one embodiment, the CEST magnetic resonance images can indicate a spatial distribution of the non-labeled therapeutic agent in the tissue. And the at least one of a therapeutic plan or therapeutic effect can be based on an assessment of the spatial distribution of the non-labeled therapeutic agent in the tissue. Further, the CEST magnetic resonance images can indicate an effective concentration of the non-labeled therapeutic agent in the tissue. And the at least one of a therapeutic plan or therapeutic effect can be based on an assessment of the effective concentration.

If drugs are directly MRI-visible, the cost (i.e., time and money) of clinical trials can be significantly reduced because the use of additional imaging agents can be, if not completely eliminated, minimized. The efficacy of an anticancer drug could be elevated only among patients who have shown effective tumor uptake of the drug, so-called stratification. And if a drug fails, it will be easy to know whether it is due to the insufficient toxicity of the drug against cancer cells, or due to the ineffectiveness of delivery. As such, the success rate of phase I trials (safety studies) can be increased because the evaluation will be carried out only on cancer patients who show effective drug accumulation and penetration. For the same reason, the success rates of phase II/III trials will also be increased and the duration will be decreased.

In addition to providing information directly about drug delivery, drugCEST can also be used according to some embodiments of the current invention to predict drug resistance by assessing the activity of certain enzymes directly related to drug resistance. Thus, one embodiment can further include acquiring a plurality of CEST magnetic resonance images of enzymes linked to the non-labeled therapeutic agent. This embodiment can further in include assessing activity of the enzymes based on the plurality of magnetic resonance images. This embodiment can further include predicting resistance to the non-labeled therapeutic agent based on the activity of the enzymes. For example, two enzymes, cytidine deaminase (CDA) and deoxycytidine kinase (dCk), have been reported to be highly related to the drug resistance of cancer cells to gemcitabine¹³. CDA catalyzes the removal of the amino group (deamination) from gemcitabine, and thus, deactivates the drugs¹⁴. Conversely, dCk is essential for the action of gemcitabine by phosphorylating the drug from the prodrug form into its active form¹⁵. We have established a CEST MRI technique platform in previous studies for assessing the activity of enzyme such as cytosine deaminase¹⁶, protein kinase¹⁷ and thymidine kinase¹⁸, the entire contents of which are incorporated herein by reference. It therefore is possible to use CEST MRI to detect CDA by observing the subsequent decrease in drugCEST, and to detect dCk by assessing the enhanced retention of drugCEST in tumor (the phosphorylation may not change the amino CEST signal as evidenced by our previous studies and the CEST signal of the phosphorylated form of fludarabine listed in Table 1). Consequently, drugCEST MRI can also be used to detect the enzyme activity that is highly related to drug resistance simply by using the drug (e.g., gemcitabine) as the imaging probe, which is also of important clinical significance.

Directly visualizing and quantifying drugs with MRI can also accelerate the pre-clinical development and clinical use of new strategies aiming at improving the targeted drug delivery. For example, it has been of vast research interest to develop nano-sized drug carriers to improve the therapeutic index and to reduce the systemic toxicity of small chemotherapeutic agents. However, while there are more than ten nanoparticulate anticancer therapeutics on the market¹⁹⁻²¹, the overall improvement in the survival rate remains modest²²⁻²⁶. It is now accepted that the enhanced permeability and retention (EPR) effect, which has been believed to be the key mechanism for passive targeting of tumor by macromolecular drug carriers, is often overrated²⁷. This formidable hurdle, however, can be at least partially overcome by using drugCEST MRI technology to pre-screen patients to determine which individuals might benefit from nanoparticulate therapeutics. In addition, there is an ongoing interest in optimizing the surface physical properties of nanoparticles, developing active targeting, or adding moieties on the surface of nanoparticles for cell internalization or stimulus response. The proposed drugCEST technology can directly monitor drug delivery and subsequent release, providing information for the rationale evolution/optimization of the nanoparticles. Unlike the radioactive labeling methods (¹⁸F or ¹⁴C), drugCEST MRI can be performed repetitively in an extended time window. Compared to the approaches based on additional labeling, information provided by drugCEST MRI is directly from the drugs and thus more accurate.

Recently, there is a growing interest to develop interventions that can significantly improve the therapeutic index either by selectively increasing the tumor vascular permeability or by normalizing the extracellular matrix to reduce the interstitial fluid pressure (IFP)²⁰. For instance, evidence from a number of recent studies^{28,29,30} revealed that co-injection of the pro-inflammatory cytokine Tumor Necrosis Factor-α (TNF-α) can greatly improve the tumor-selective accumulation of liposomal drugs by augmenting the enhanced permeability and retention (EPR) effect. Unlike the approaches based on macromolecular agents, drugCEST is able to provide direct and dynamic information about the penetration of drugs as the consequence of a treatment that targets to improve the therapeutic index. In addition, drugCEST MRI is also able to measure pH, which can indirectly reveal the location of the administered drugs as pH correlates well with the pathology of tumors. For example, when drugs remain in capillaries, they are surrounded by blood with a narrow pH range of 7.35 to 7.45 and, in contrast, if they penetrate to the poorly perfused regions of tumor, they will likely have a pH range of 6.0-6.5. In the previous studies, we have established a concentration-independent approach for accurately determining pH simply by calculating the ratio of CEST signals from two different types of exchangeable protons on the same molecule³¹⁻³³, the entire contents of which are incorporated herein by reference. As many drugs have multiple types of exchangeable protons, it is, therefore, possible to use drugCEST—in addition to assessing drug concentration—to measure the pH where drugs are located.

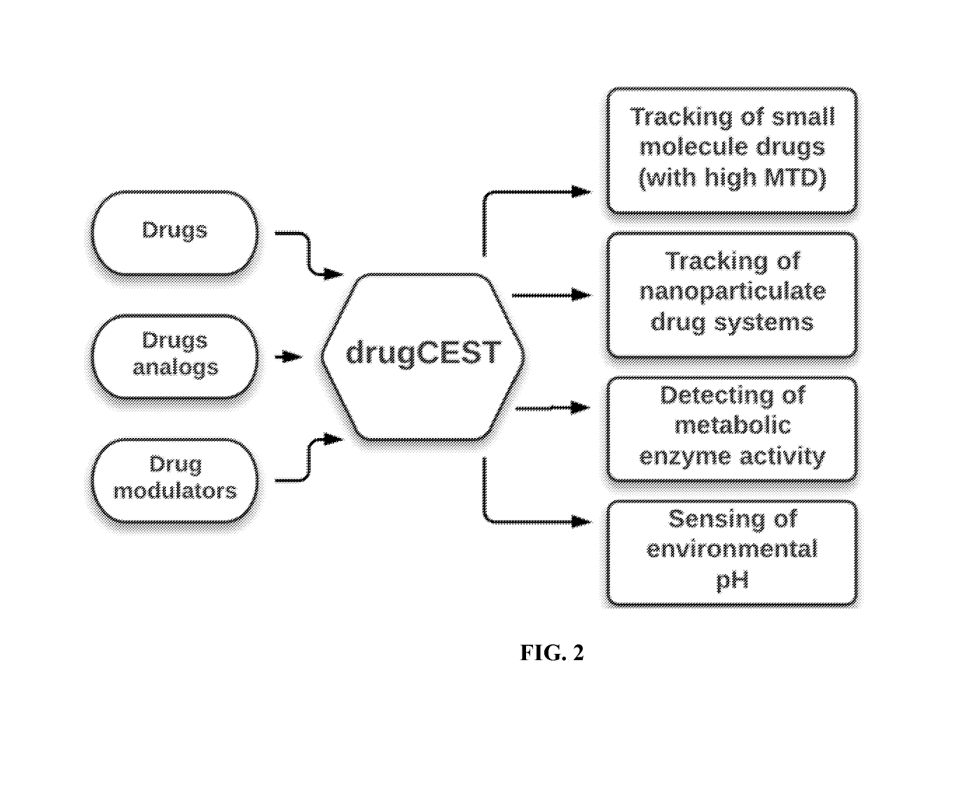

Collectively, the successful establishment of a highly MRI-based technology to directly and noninvasively image drugs according to some embodiments of the current invention may immensely benefit the clinical treatment of cancer and the preclinical development of new drugs and nanoparticulate therapeutics. Some potential roles of drugCEST MRI according to some embodiments of the current invention are illustrated in FIG. 2.

Some aspects of the current invention are directed to the following:
1. A label-free imaging approach to “see” drugs directly, enabling patient stratification without the use of imaging agents. A novel feature of such an embodiment lies in that it uses a “label-free” (i.e., not radioactive, and not paramagnetic- or super-paramagnetic-based) approach to detect and quantify the administered drugs. This has never been demonstrated except by spectroscopic methods (both ¹H MRS and ¹⁹F MRS), which, however, often suffer from low sensitivity and low spatiotemporal resolution. In contrast, our approach employs the CEST strategy to amplify the small signal from low-concentration drugs, enabling the direct detection of drugs with MRI.
2. An approach that is suitable for a broad array of drugs. Since a requirement for drugCEST is to have exchangeable proton with an appropriate exchange rate at a sufficient concentration, and since many drugs contain labile protons such as NH, NH₂ and OH, our approach, in principle, can be expanded to many categories of drugs. We have tested several antimetabolites and we anticipate that our drugCEST technology is suitable for many drugs, drug analogs, drug modulators and even drugs carriers (dextran, dendrimer, etc.). In addition, we also foresee that the principle behind this technique can be expanded to drugs designed to treat other diseases.
3. An approach that exploits drugs themselves as biomarkers to predict drug action and therapeutic outcomes. Another innovation is the combined capability of detecting concentration, pH, and metabolic enzyme activity. Consequently, drugCEST can be used as a predictive marker for the therapeutic outcome.
4. An approach that also exploits nontoxic drug analogs as a predictor for the behavior of the corresponding drugs. In addition to the drugs, whose applicability may be limited by their potential toxicity, we will also employ drug analogs, the non-toxic compounds, which have a chemical structure similar to the drugs. As such, we can use much higher doses to achieve a much stronger CEST signal. Using nontoxic drug analogs to “rehearse” the drug delivery and drug metabolism provides a new means to safely stratify patients appropriate for a treatment.
5. A technology that can be synergized with nanomedicine to transform currently available therapeutics directly into theranostics. We can use nanoparticles, especially those have been approved by FDA, to improve the sensitivity of drugCEST as an alternative to some embodiments. Consequently, the nanocarriers encapsulated with MRI-visible drugs now are not only able to deliver drugs (treat the tumor) but also to “see” the drug delivery to tumors. Compared to other previously reported theranostic systems, our approach requires no extra contrast agent, therefore will have minimal barriers to be used in patients.
6. A technology that can be combined with other MRI techniques to detect multiple targets simultaneously in the course of chemotherapy. CEST holds great promise for its ability to be selectively turned on and off at will by turning radiofrequency pulses on and off^{34,35}, the ability to combine with other MRI contrast,^{36,37} and, most importantly, the ability to simultaneously detect multiple CEST agents by their specific CEST frequency, in a way similar to optical imaging (so-called multi-frequency or multi-colored detection)^{35,38,39}. These unique features thus allow combining drugCEST MRI with functional and morphological MRI technologies, and even other MR molecular imaging, to fully understand the biological effects of chemotherapy.

### Overview of In Vitro and In Vivo Data

### 1. In Vitro Phantom Studies

We have discovered that 5-fluorocytosine 5-FC, an antifungal drug and also a prodrug (compounds that can be converted to effective drugs) for cancer gene therapy, has a strong CEST signal, allowing us to detect the presence and metabolism of the drug without the use of imaging agent⁹. To the best of our knowledge, this is the first demonstration of label-free detecting of a drug directly by its CEST contrast. Other drugs, including prodrugs and drug modulators, as well as the drug carriers, are intended to be included within the general scope of this invention. Many of them have exchangeable protons such as OH, NH and NH₂ (FIG. 1). Thus, in one embodiment, the water exchangeable proton corresponds to at least one of an OH, NH2 or NH group. The water-exchangeable proton can include at least one of an OH, NH2 or NH group. Other antitumor categories and the biochemical drug modulators (a non-toxic agent that can enhance the effect of drugs when used together, but has no effect when used alone) as listed in Table 1 are also intended to be within the scope of the current invention.

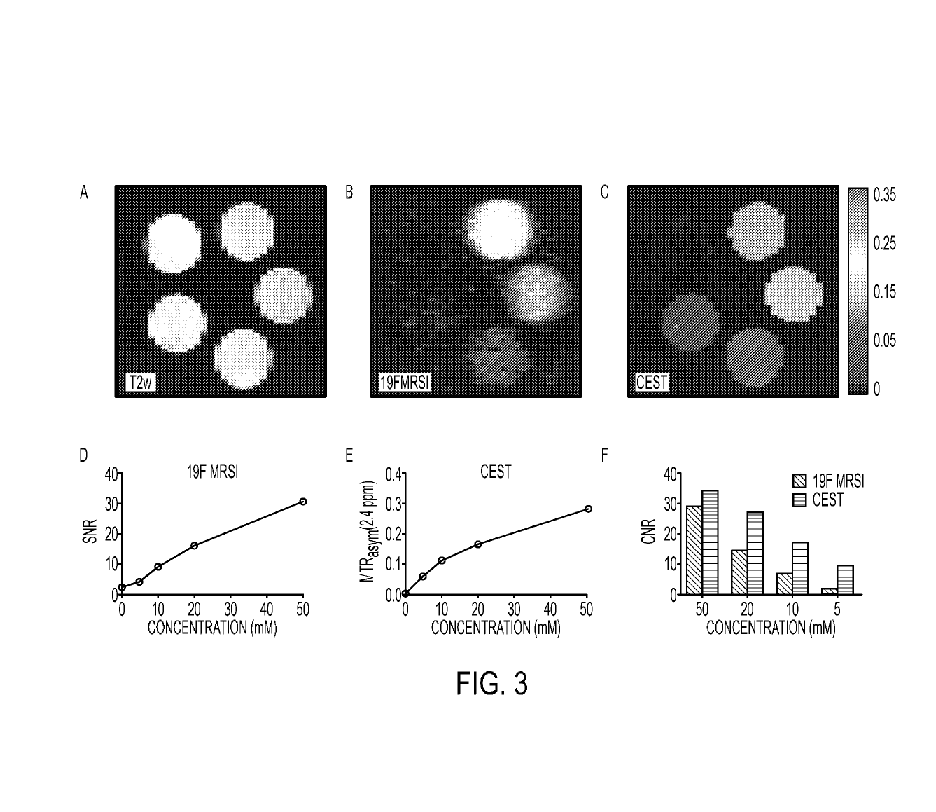

In principle, drugCEST can be directly applicable to drugs that are being used at a relatively high dose in patients. For instance, in the regime of high-dose Cytarabine (araC) therapy for leukemia, it was reported that no noticeable cerebellar toxicity was found among patients who received repetitive infusions of araC (single dose, 3 g/m² over three hours) for up to eight doses (total dose, 24 to 30 g/m²)⁴⁷. As a reference, a single dose of 3 g/m² corresponds to 80 mg/kg and 1000 mg/kg in humans and in mice respectively, using a body weight of 60 and 0.02 kg and a body surface area of 1.6 and 0.007 m² respectively⁴⁸. Another widely used anticancer drug gemcitabine has a clinically suggested dose of 1000 mg/m² for treating of a variety of cancer types (equivalent mouse dose=333 mg/kg). In fact, it was reported that a single dose of 800 mg/kg (i.p.) could result in an accumulation of gemcitabine up to several mM in experimental murine hepatomas, allowing the detection the drug using ¹⁹F MR spectroscopic imaging (MRSI)⁴⁹. As aforementioned, the CEST MRI generates a huge amplification of the NMR signal of a small agent, and thus, in principle, possesses a higher detectability than ¹H spectroscopic and ¹⁹F MR spectroscopic methods. Based on the literature and our preliminary in vitro result (FIG. 3, which shows that CEST MRI could generate a five-fold higher contrast-to-noise ratio (CNR) than ¹⁹F MRSI in detecting 5 mM gemcitabine at the same temporal and spatial resolution), it is possible to use drugCEST MRI to detect and map the tumor uptake of those drugs that can be administered at high doses. In some examples, to obtain possibly sufficient in vivo drug concentration, we can only evaluate drugs with a maximum tolerated dose (MTD) higher than 300 mg/m² (equivalent mouse dose>100 mg/kg) in the present study. For instance, gemcitabine, the first-line chemotherapeutic agent for pancreatic cancer, has a high MTD (2200 m^{g/m2})^{50,} therefore is selected as a model compound to be evaluated in our study.

Elements (a)-(c) of FIG. 3 show T2-weighted imaging, 19F MRSI and CEST MRI of five samples containing gemcitabine at concentration of 0, 5, 10, 20 and 50 mM respectively. Element (d) of FIG. 3 shows the mean ROI SNR of the samples assessed by 19F MRSI. Element (e) of FIG. 3 shows the mean ROI CEST contrast at 2.4 ppm of the samples. Element (f) of FIG. 3 shows the comparison of their contrast-to-noise ratio (CNR) at each concentration as opposed to the signal of PBS.

We also examined the possibility the use of nontoxic drug analogs as predictive markers for the corresponding drugs in the case that drugs cannot accumulate in the tumor at a sufficient concentration. Most anti-metabolic drugs are derived from natural metabolites. These non-toxic drug analogs have similar chemical structures with the corresponding drugs, and thus similar CEST signals, and, very often, similar pharmacokinetics and pharmacodynamics. Therefore we can use non-toxic drug analogs as the predictive markers of the delivery and metabolism of the corresponding drugs. As such, the chemotherapy can be “rehearsed” using non-toxic drug analog. Patients can be stratified and only those whose tumors are accessible by the drugs can be selected to receive the actual treatment. The advantage of using drug analogs is the possibility to use high dose administration without introducing severe adverse effects, as is the case with the drugs. Our results (Table 2) have shown the drugCEST of the representative drug analog in each category. This alternative can warrant the detectability of drugCEST even drugs cannot be directly used.

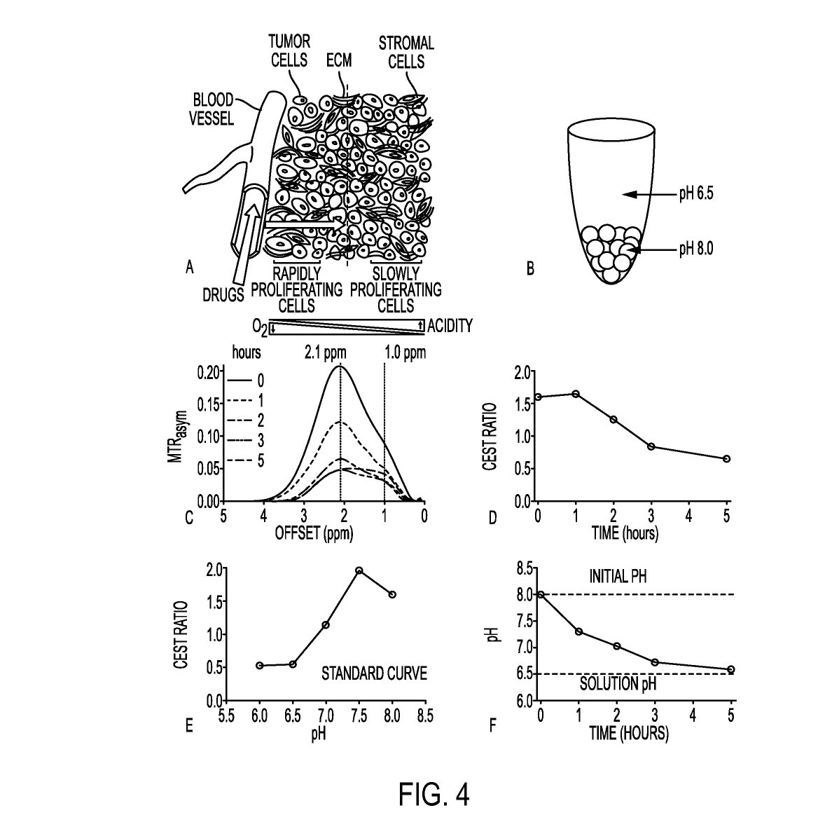

Some embodiments of the current invention can also be used to reveal the location of delivered drugs by remote sensing of pH. That is, one embodiment can include determining a pH of the region of interest based on a CEST signal of the CEST magnetic resonance images of the non-labeled therapeutic agent. The CEST signal can indicate penetration of the therapeutic agent in the region of interest. As is well-documented, pH affects the exchange rate dramatically, and consequently influences the CEST effect substantially^{26,27}. If a CEST agent possesses two or more types of exchangeable protons, and if their pH dependencies are different, pH can be estimated by the ratio of their CEST signals²⁸⁻³⁰. Luckily, many drugs have multiple types of exchangeable protons, thus allowing us to measure the pH of where drugs are located. Because pH correlates well to the physiology and pathology of tumor (Element (a) of FIG. 4), it is possible to precisely determine the location of drugs by pH. In an embodiment, the non-labeled therapeutic agent can include at least two different types of water exchangeable protons. The determining a pH step can be based on a ratio of CEST signals from the two different water exchangeable protons. For example, if the drugs remain in the capillaries, they are surrounded by blood with a narrow pH range of 7.35 to 7.45. In contrast, if the drugs are in the extra-vascular extra-cellular space of a poorly perfused region, they are likely to experience a pH range of 6.0-6.5. Therefore we expect that the drugCEST can be used to precisely determine the location of the delivered drugs if the drug has more than CEST signals and their pH dependency are different. To demonstrate it, as shown in element (b) of FIG. 4, we measured the drugCEST signals of gemcitabine at 2.1 ppm (NH2) and 1.0 ppm (OH) at different time points after immersing liposomal gemcitabine (initial intra-liposomal pH=8.0) in pH 6.5 solution. The pH values at each time points were calculated by the ratio of the two CEST signals using a protocol that we previously published^{26,28} (Elements (c)-(f) of FIG. 4), clearly showing the equilibrium between intra- and extra-liposomal pH's after three hours. Consequently, the environmental pH can be assessed by measuring the drugCEST signal of encapsulated drugs.

Element (a) of FIG. 4 shows an illustration of the tumor anatomy-related pH variation. Element (b) of FIG. 4 shows an experiment set up for measuring pH using liposomal gemcitabine. Element (c) of FIG. 4 shows measured CEST signals and FIG. 4(d) shows the CEST ratio of two CEST signals at different time points. According to a standard pH response curve measured previously in Element (e) of FIG. 4, the pH of liposomal pH was calculated in Element (f) of FIG. 4.

2. In Vivo Animal Studies

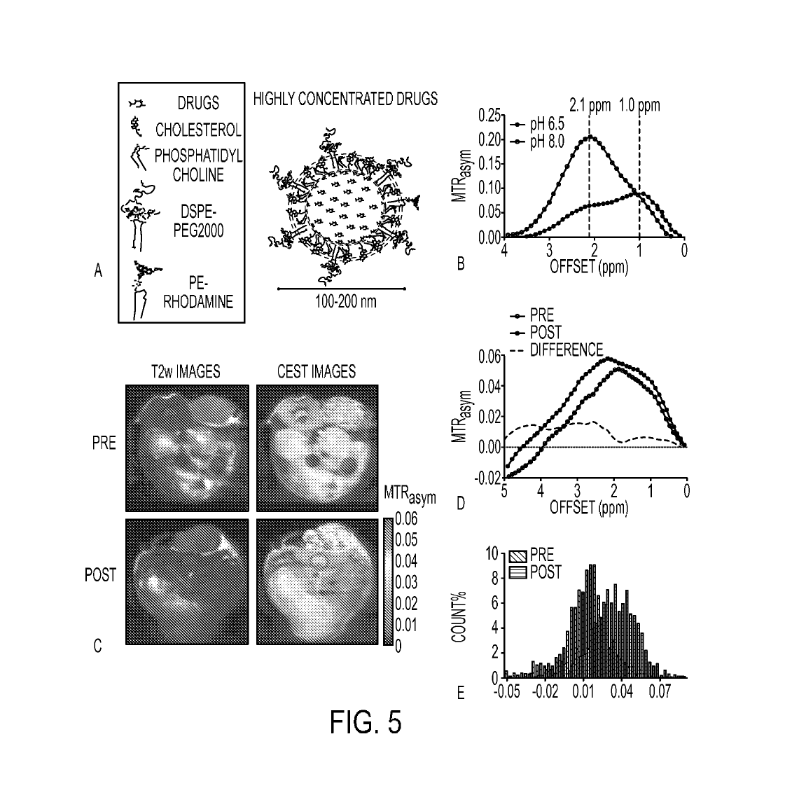

According to an embodiment of the current invention, when drugs fail to generate detectable drugCEST due to low concentration, we can also use nanocarriers such as liposomes to push the detection limit. As we and others have demonstrated^{38,51-53}, encapsulating CEST agents into liposomes could markedly improve the detection limit from mM(per molecule) to nM (per particle)³⁸, and has enabled applications in experimental animal models⁵⁴. Some examples (FIG. 5) also showed that liposomal gemcitabine could be readily detected in tumors, indicating that embodiments of our invention can still be useful to the pre-clinical development and clinical use of nanoparticulate chemotherapeutic agents even if directly detecting drugs in their free form fails. Using nanoscale drug carriers to boost the sensitivity of drugCEST is in line with the use of nanoparticulate therapeutics, and, very recently, theranostic (therapeutics and diagnostics) nanoparticles⁵⁵. There are more than 45 nanoparticulate drug formulations that have been clinically approved and at least 200 products currently in Phase I-III clinical trials¹⁹. For example, Depocyt, a liposomal formulation of Cytarabine (araC, FIG. 1e left) was approved in 1999 for treating neoplastic meningitis and lymphomatous meningitis⁵⁶. Therefore, it is quite reasonable to apply the drugCEST technology directly on the clinically used nanoparticulate drug systems to stratify patients and enable personalized medicine, or on those under pre-clinical evaluation to accelerate their clinical translation. Another advantage of using nanocarriers is that, even when a drug does not have drugCEST, we still can possibly use the inherent CEST signal carried by some drug carriers to realize our goal of label-free detection of drug delivery. For example, as early as in 2001, van Zijl and his colleagues reported that several polymer gene delivery systems could be detected by CEST MRI⁵⁷. In another ongoing project, the CEST signal of dextran, a clinically used nanoparticle⁵⁸ is being investigated. Therefore ‘label-free’ detection of these drug delivery systems is also feasible.

Element (a) of FIG. 5 shows an illustrtution of a drug-encapsulated liposome system. Element (b) of FIG. 5 shows the CEST signal of 20 nM (per particle) gemcitabine-liposomes at two different pH in PBS using standard CEST MRI protocol with B1=3.6 μT and 3 second duration. Element (c) of FIG. 5 shows the first in vivo demonsration of detection of drug-carried nanoparticles in vivo without the use of additonal imaging agents. The bottom panels show the T2w image and CEST map at ˜3.2 ppm (only tumor region is shown, and 3.2 ppm is the offset that the maximium CEST signal could be detected for gemcitabine at low pH) at 5 hours after the liposomes injection, clearly exhibiting a striking increase of CEST contrast across the tumor region compared to that before injection (top panel). Elements (d)-(e) of FIG. 5 show the quantifiation of uptake of gemcitabine by comparing either the mean MTR_{asym} plots of whole tumor regions (d) or histogram analysis of the MTR_{asy m} values of pixels within tumor regions (e) before and after injection of liposomes. Note that the difference of MTR_{sym} plots before and after injection showed a different CEST pattern as compared to those in PBS solution. This is likely attirbuted to in vivo MT effect and endogenous CEST effects, which can compete with the gemcitabine for water exchange and subsequently affect the CEST pattern. Regardless of this discrepancy, the in vivo CEST MRI showed a steadily detectable CEST signal (˜2%) due to the accumulation of drug carriers.

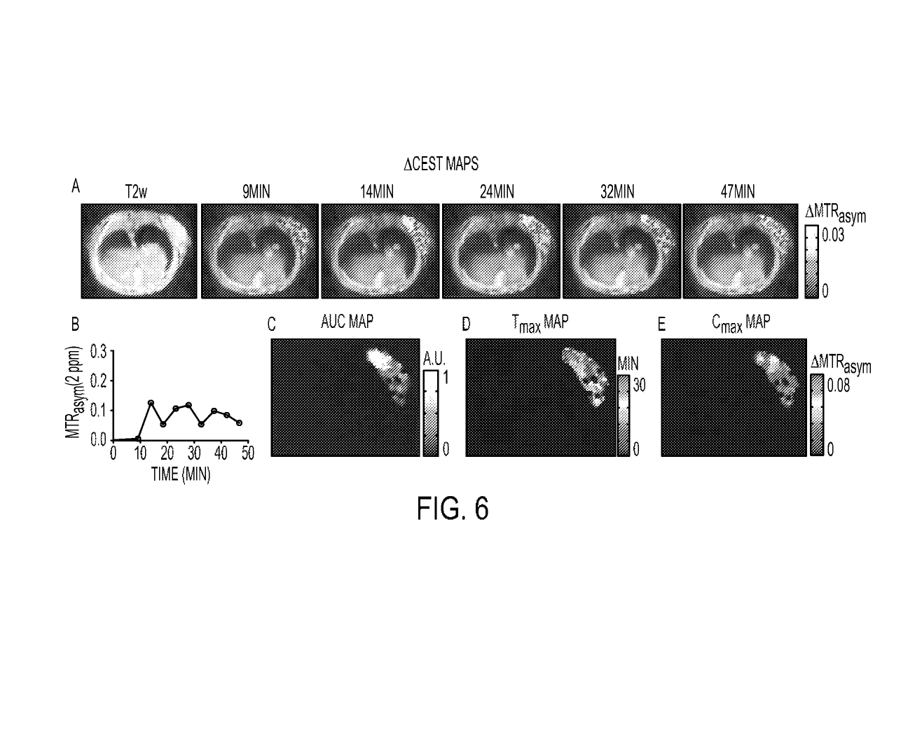

Element (a) of FIG. 6 shows a ΔCEST (i.e. change in CEST) MRI contrast maps at 2.3 ppm over a period of 50 minutes after the injection of 500 mg/kg gemcitabine into the tail vein of the mouse. Note that only the CEST contrast within the tumor region is shown. Element (b) of FIG. 6 shows a dynamic change in CEST contrast for the whole tumor. Elements (c)-(d) of FIG. 6 shows the calculated pharmacokinetic parametric maps: Area Under Curve (AUC), the time to maximal contrast enhancement (Tmax); and the maximal CEST enhancement (Cmax).

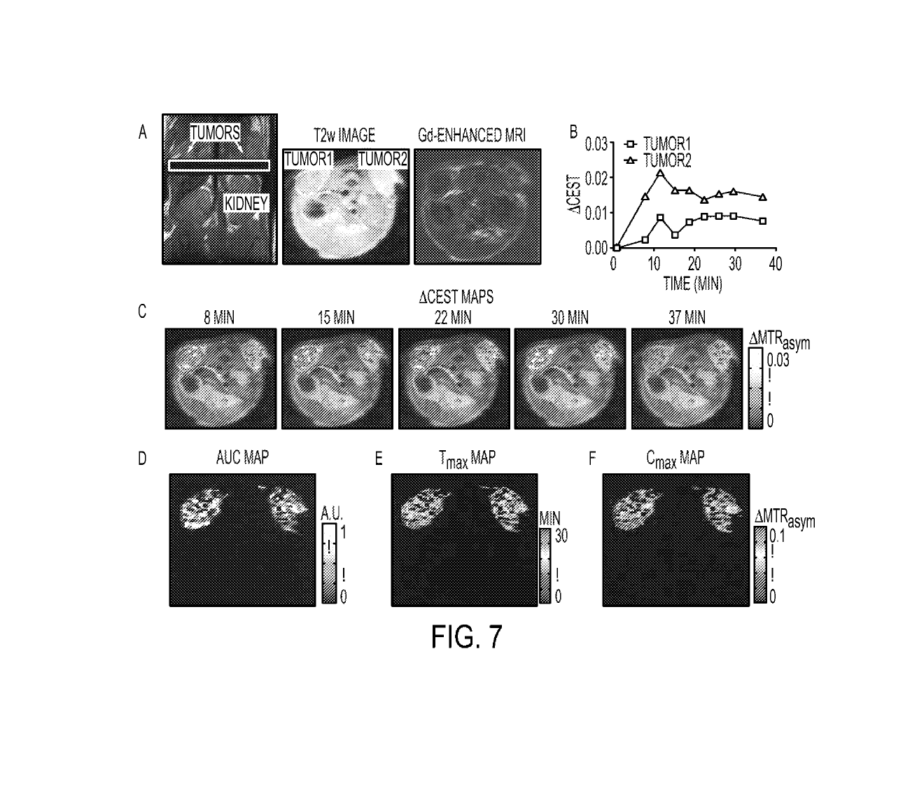

Element (a) of FIG. 7 shows MRI images. Element (b) of FIG. 7 shows a plot displaying the dynamic change in the CEST signal of the tumors at different post-injection times. Element (c) of FIG. 7 shows the ΔCEST maps at different post-injection times. Elements (c)-(d) of FIG. 7 show the calculated pharmacokinetic parametric maps: Area Under Curve (AUC), the maximum intra-tumoral dFdC concentration (Cmax) and the time at which the Cmax is observed (Tmax).

### REFERENCES

1. van Zijl, P. C. & Yadav, N. N. Chemical exchange saturation transfer (cest): What is in a name and what isn't? Magn. Reson. Med. 65, 927-948 (2011).
2. Liu, G., Gilad, A. A., Bulte, J. W., van Zijl, P. C. & McMahon, M. T. High-throughput screening of chemical exchange saturation transfer mr contrast agents. Contrast Media Mol. Imaging 5, 162-170 (2010).
3. Tredan, O., Galmarini, C. M., Patel, K. & Tannock, I. F. Drug resistance and the solid tumor microenvironment. J. Natl. Cancer Inst. 99, 1441-1454 (2007).
4. Olive, K. P., Jacobetz, M. A., Davidson, C. J., Gopinathan, A., McIntyre, D., Honess, D., Madhu, B., Goldgraben, M. A., Caldwell, M. E., Allard, D., Frese, K. K., Denicola, G., Feig, C., Combs, C., Winter, S. P., Ireland-Zecchini, H., Reichelt, S., Howat, W. J., Chang, A., Dhara, M., Wang, L., Ruckert, F., Grutzmann, R., Pilarsky, C., Izeradjene, K., Hingorani, S. R., Huang, P., Davies, S. E., Plunkett, W., Egorin, M., Hruban, R. H., Whitebread, N., McGovern, K., Adams, J., Iacobuzio-Donahue, C., Griffiths, J. & Tuveson, D. A. Inhibition of hedgehog signaling enhances delivery of chemotherapy in a mouse model of pancreatic cancer. Science 324, 1457-1461 (2009).
5. Ginsburg, G. S. & McCarthy, J. J. Personalized medicine: Revolutionizing drug discovery and patient care. TRENDS in Biotechnology 19, 491-496 (2001).
6. Nunn, A. D. Update: Molecular imaging and personalized medicine: An uncertain future. Cancer Biother. Radiopharm. 22, 722-739 (2007).
7. Caride, V. J., Sostman, H. D., Winchell, R. J. & Gore, J. C. Relaxation enhancement using liposomes carrying paramagnetic species. Magn. Reson. Imaging 2, 107-112 (1984).
8. Devoisselle, J. M., Vion-Dury, J., Galons, J. P., Confort-Gouny, S., Coustaut, D., Canioni, P. & Cozzone, P. J. Entrapment of gadolinium-dtpa in liposomes. Characterization of vesicles by p-31 nmr spectroscopy. Invest. Radiol. 23, 719-724 (1988).
9. Zhang, J. Q., Zhang, Z. R., Yang, H., Tan, Q. Y., Qin, S. R. & Qiu, X. L. Lyophilized paclitaxel magnetoliposomes as a potential drug delivery system for breast carcinoma via parenteral administration: In vitro and in vivo studies. Pharm. Res. 22, 573-583 (2005).
10. Gigli, M., Doglia, S. M., Millot, J. M., Valentini, L. & Manfait, M. Quantitative study of doxorubicin in living cell nuclei by microspectrofluorometry. Biochim. Biophys. Acta 950, 13-20 (1988).
11. Stevens, A., Morris, P., Iles, R., Sheldon, P. & Griffiths, J. 5-fluorouracil metabolism monitored in vivo by 19f nmr. Br. J. Cancer 50, 113 (1984).
12. Ward, K. M., Aletras, A. H. & Balaban, R. S. A new class of contrast agents for mri based on proton chemical exchange dependent saturation transfer (cest). J. Magn. Reson. 143, 79-87 (2000).
13. Bouffard, D. Y., Laliberté, J. & Momparler, R. L. Kinetic studies on 2′, 2′-difluorodeoxycytidine (gemcitabine) with purified human deoxycytidine kinase and cytidine deaminase. Biochem. Pharmacol. 45, 1857-1861 (1993).
14. Gilbert, J. A., Salavaggione, O. E., Ji, Y., Pelleymounter, L. L., Eckloff, B. W., Wieben, E. D., Ames, M. M. & Weinshilboum, R. M. Gemcitabine pharmacogenomics: Cytidine deaminase and deoxycytidylate deaminase gene resequencing and functional genomics. Clin. Cancer Res. 12, 1794-1803 (2006).
15. Sebastiani, V., Ricci, F., Rubio-Viqueira, B., Kulesza, P., Yeo, C. J., Hidalgo, M., Klein, A., Laheru, D. & Iacobuzio-Donahue, C. A. Immunohistochemical and genetic evaluation of deoxycytidine kinase in pancreatic cancer: Relationship to molecular mechanisms of gemcitabine resistance and survival. Clin. Cancer Res. 12, 2492-2497 (2006).
16. Liu, G., Liang, Y., Bar-Shir, A., Chan, K. W., Galpoththawela, C. S., Bernard, S. M., Tse, T., Yadav, N. N., Walczak, P., McMahon, M. T., Bulte, J. W., van Zijl, P. C. & Gilad, A. A. Monitoring enzyme activity using a diamagnetic chemical exchange saturation transfer magnetic resonance imaging contrast agent. J. Am. Chem. Soc. 133, 16326-16329 (2011).
17. Airan, R. D., Bar-Shir, A., Liu, G., Pelled, G., McMahon, M. T., van Zijl, P. C., Bulte, J. W. & Gilad, A. A. Mri biosensor for protein kinase a encoded by a single synthetic gene. Magn. Reson. Med. 68, 1919-1923 (2012).
18. Bar-Shir, A., Liu, G., Liang, Y., Yadav, N. N., McMahon, M. T., Walczak, P., Nimmagadda, S., Pomper, M. G., Tallman, K. A., Greenberg, M. M., van Zijl, P. C., Bulte, J. W. & Gilad, A. A. Transforming thymidine into a magnetic resonance imaging probe for monitoring gene expression. J. Am. Chem. Soc. 135, 1617-1624 (2013).
19. Davis, M. E., Chen, Z. G. & Shin, D. M. Nanoparticle therapeutics: An emerging treatment modality for cancer. Nat. Rev. Drug Discov. 7, 771-782 (2008).
20. Jain, R. K. & Stylianopoulos, T. Delivering nanomedicine to solid tumors. Nature reviews. Clinical oncology 7, 653-664 (2010).
21. Zhang, L., Gu, F. X., Chan, J. M., Wang, A. Z., Langer, R. S. & Farokhzad, O. C. Nanoparticles in medicine: Therapeutic applications and developments. Clin. Pharmacol. Ther. 83, 761-769 (2008).
22. Gill, P. S., Wernz, J., Scadden, D. T., Cohen, P., Mukwaya, G. M., von Roenn, J. H., Jacobs, M., Kempin, S., Silverberg, I. & Gonzales, G. Randomized phase iii trial of liposomal daunorubicin versus doxorubicin, bleomycin, and vincristine in aids-related kaposi's sarcoma. J. Clin. Oncol. 14, 2353-2364 (1996).
23. Northfelt, D. W., Dezube, B. J., Thommes, J. A., Miller, B. J., Fischl, M. A., Friedman-Kien, A., Kaplan, L. D., Du Mond, C., Mamelok, R. D. & Henry, D. H. Pegylated-liposomal doxorubicin versus doxorubicin, bleomycin, and vincristine in the treatment of aids-related kaposi's sarcoma: Results of a randomized phase iii clinical trial. J. Clin. Oncol. 16, 2445-2451 (1998).
24. Gordon, A. N., Fleagle, J. T., Guthrie, D., Parkin, D. E., Gore, M. E. & Lacave, A. J. Recurrent epithelial ovarian carcinoma: A randomized phase iii study of pegylated liposomal doxorubicin versus topotecan. J. Clin. Oncol. 19, 3312-3322 (2001).
25. O'brien, M., Wigler, N., Inbar, M., Rosso, R., Grischke, E., Santoro, A., Catane, R., Kieback, D., Tomczak, P. & Ackland, S. Reduced cardiotoxicity and comparable efficacy in a phase iii trial of pegylated liposomal doxorubicin hcl (caelyx™/doxil®) versus conventional doxorubicin for first-line treatment of metastatic breast cancer. Ann. Oncol. 15, 440-449 (2004).
26. Gradishar, W. J., Tjulandin, S., Davidson, N., Shaw, H., Desai, N., Bhar, P., Hawkins, M. & O'Shaughnessy, J. Phase iii trial of nanoparticle albumin-bound paclitaxel compared with polyethylated castor oil-based paclitaxel in women with breast cancer. J. Clin. Oncol. 23, 7794-7803 (2005).
27. Lammers, T., Kiessling, F., Hennink, W. E. & Storm, G. Drug targeting to tumors: Principles, pitfalls and (pre-) clinical progress. J. Control. Release 161, 175-187 (2012).
28. Qiao, Y., Huang, X., Nimmagadda, S., Bai, R., Staedtke, V., Foss, C. A., Cheong, I., Holdhoff, M., Kato, Y., Pomper, M. G., Riggins, G. J., Kinzler, K. W., Diaz, L. A., Jr., Vogelstein, B. & Zhou, S. A robust approach to enhance tumor-selective accumulation of nanoparticles. Oncotarget 2, 59-68 (2011).
29. Jain, R. K., Duda, D. G., Clark, J. W. & Loeffler, J.S. Lessons from phase iii clinical trials on anti-vegf therapy for cancer. Nat. Clin. Pract. Oncol. 3, 24-40 (2006).
30. Matsumura, Y. & Maeda, H. A new concept for macromolecular therapeutics in cancer chemotherapy: Mechanism of tumoritropic accumulation of proteins and the antitumor agent smancs. Cancer Res. 46, 6387-6392 (1986).
31. Liu, G., Li, Y., Sheth, V. R. & Pagel, M. D. Imaging in vivo extracellular ph with a single paramagnetic chemical exchange saturation transfer magnetic resonance imaging contrast agent.Mol. Imaging 11, 47-57 (2012).
32. Ward, K. M. & Balaban, R. S. Determination of ph using water protons and chemical exchange dependent saturation transfer (cest). Magn. Reson. Med. 44, 799-802 (2000).
33. Aime, S., Barge, A., Delli Castelli, D., Fedeli, F., Mortillaro, A., Nielsen, F. U. & Terreno, E. Paramagnetic lanthanide(iii) complexes as ph-sensitive chemical exchange saturation transfer (cest) contrast agents for mri applications. Magn. Reson. Med. 47, 639-648 (2002).
34. Zhang, S., Merritt, M., Woessner, D. E., Lenkinski, R. E. & Sherry, A. D. Paracest agents: Modulating mri contrast via water proton exchange. Acc Chem Res 36, 783-790 (2003).
35. Aime, S., Carrera, C., Delli Castelli, D., Geninatti Crich, S. & Terreno, E. Tunable imaging of cells labeled with mri-paracest agents. Angew. Chem. Int. Ed. 44, 1813-1815 (2005).
36. Aime, S., Castelli, D. D., Lawson, D. & Terreno, E. Gd-loaded liposomes as tl, susceptibility, and cest agents, all in one. J. Am. Chem. Soc. 129, 2430-2431 (2007).
37. Gilad, A. A., van Laarhoven, H. W., McMahon, M. T., Walczak, P., Heerschap, A., Neeman, M., van Zijl, P. C. & Bulte, J. W. Feasibility of concurrent dual contrast enhancement using cest contrast agents and superparamagnetic iron oxide particles. Magn. Reson. Med. 61, 970-974 (2009).
38. Liu, G., Moake, M., Har-el, Y. E., Long, C. M., Chan, K. W., Cardona, A., Jamil, M., Walczak, P., Gilad, A. A., Sgouros, G., van Zijl, P. C., Bulte, J. W. & McMahon, M.T. In vivo multicolor molecular mr imaging using diamagnetic chemical exchange saturation transfer liposomes. Magn. Reson. Med. 67, 1106-1113 (2012).
39. McMahon, M. T., Gilad, A. A., DeLiso, M. A., Berman, S. M., Bulte, J. W. & van Zijl, P. C. New “multicolor” polypeptide diamagnetic chemical exchange saturation transfer (diacest) contrast agents for mri. Magn. Reson. Med. 60, 803-812 (2008).
40. Feinberg, B., Kurzrock, R., Talpaz, M., Blick, M., Saks, S. & Gutterman, J.U. A phase i trial of intravenously-administered recombinant tumor necrosis factor-alpha in cancer patients. J. Clin. Oncol. 6, 1328-1334 (1988).

41. Li, W., Zhang, Z., Nicolai, J., Yang, G. Y., Omary, R. A. & Larson, A. C. Magnetization transfer mri in pancreatic cancer xenograft models. Magn. Reson. Med. 68, 1291-1297 (2012).
42. Grimm, J., Potthast, A., Wunder, A. & Moore, A. Magnetic resonance imaging of the pancreas and pancreatic tumors in a mouse orthotopic model of human cancer. Int. J. Cancer 106, 806-811 (2003).
43. Jones, C. K., Schlosser, M. J., van Zijl, P. C., Pomper, M. G., Golay, X. & Zhou, J. Amide proton transfer imaging of human brain tumors at 3t. Magn. Reson. Med. 56, 585-592 (2006).
44. Zhou, J., Blakeley, J. O., Hua, J., Kim, M., Laterra, J., Pomper, M. G. & van Zijl, P. C. Practical data acquisition method for human brain tumor amide proton transfer (apt) imaging. Magn. Reson. Med. 60, 842-849 (2008).
45. Salhotra, A., Lal, B., Laterra, J., Sun, P. Z., van Zijl, P. & Zhou, J. Amide proton transfer imaging of 91 gliosarcoma and human glioblastoma xenografts. NMR Biomed. 21, 489-497 (2008).
46. Zhou, J., Lal, B., Wilson, D. A., Laterra, J. & van Zijl, P. C. Amide proton transfer (apt) contrast for imaging of brain tumors. Magn. Reson. Med. 50, 1120-1126 (2003).
47. Herzig, R. H., Hines, J., Herzig, G., Wolff, S., Cassileth, P., Lazarus, H., Adelstein, D., Brown, R., Coccia, P. & Strandjord, S. Cerebellar toxicity with high-dose cytosine arabinoside. J. Clin. Oncol. 5, 927-932 (1987).
48. Reagan-Shaw, S., Nihal, M. & Ahmad, N. Dose translation from animal to human studies revisited. FASEB J. 22, 659-661 (2008).
49. Cron, G. O., Beghein, N., Ansiaux, R., Martinive, P., Feron, O. & Gallez, B. 19f nmr in vivo spectroscopy reflects the effectiveness of perfusion-enhancing vascular modifiers for improving gemcitabine chemotherapy. Magn. Reson. Med. 59, 19-27 (2008).
50. Touroutoglou, N., Gravel, D., Raber, M., Plunkett, W. & Abbruzzese, J. Clinical results of a pharmacodynamically-based strategy for higher dosing of gemcitabine in patients with solid tumors. Ann. Oncol. 9, 1003-1008 (1998).
51. Aime, S., Delli Castelli, D. & Terreno, E. Highly sensitive mri chemical exchange saturation transfer agents using liposomes. Angew. Chem. 117, 5649-5651 (2005).
52. Castelli, D. D., Terreno, E., Longo, D. & Aime, S. Nanoparticle-based chemical exchange saturation transfer (cest) agents. NMR Biomed. 26, 839-849 (2013).
53. Winter, P.M. Magnetic resonance chemical exchange saturation transfer imaging and nanotechnology. Wiley Interdiscip. Rev. Nanomed. Nanobiotechnol. 10.1002/wnan.1167(2012).
54. Chan, K. W., Liu, G., Song, X., Kim, H., Yu, T., Arifin, D. R., Gilad, A. A., Hanes, J., Walczak, P., van Zijl, P.C., Bulte, J. W. & McMahon, M. T. Mri-detectable ph nanosensors incorporated into hydrogels for in vivo sensing of transplanted-cell viability. Nat. Mater. 12, 268-275 (2013).
55. Sumer, B. & Gao, J. Theranostic nanomedicine for cancer. Nanomed. 3, 137-140 (2008).
56. Glantz, M. J., LaFollette, S., Jaeckle, K. A., Shapiro, W., Swinnen, L., Rozental, J. R., Phuphanich, S., Rogers, L. R., Gutheil, J. C., Batchelor, T., Lyter, D., Chamberlain, M., Maria, B. L., Schiffer, C., Bashir, R., Thomas, D., Cowens, W. & Howell, S. B. Randomized trial of a slow-release versus a standard formulation of cytarabine for the intrathecal treatment of lymphomatous meningitis. J. Clin. Oncol. 17, 3110-3116 (1999).
57. Goffeney, N., Bulte, J. W., Duyn, J., Bryant, L. H., Jr. & van Zijl, P.C. Sensitive nmr detection of cationic-polymer-based gene delivery systems using saturation transfer via proton exchange. J. Am. Chem. Soc. 123, 8628-8629 (2001).
58. Mitra, S., Gaur, U., Ghosh, P. & Maitra, A. Tumour targeted delivery of encapsulated dextran-doxorubicin conjugate using chitosan nanoparticles as carrier. J. Control. Release 74, 317-323 (2001).
59. Liu, G., Chan, K. W., Song, X., Zhang, J., Gilad, A. A., Bulte, J. W., van Zijl, P. C. & McMahon, M. T. Normalized magnetization ratio (nomar) filtering for creation of tissue selective contrast maps. Magn. Reson. Med. 69, 516-523 (2013).
60. Xu, J., Yadav, N. N., Bar-Shir, A., Jones, C. K., Chan, K. W., Zhang, J., Walczak, P., McMahon, M. T. & van Zijl, P. C. Variable delay multi-pulse train for fast chemical exchange saturation transfer and relayed-nuclear overhauser enhancement mri. Magn. Reson. Med. 10.1002/mrm.24850 (2013).
61. Yadav, N. N., Jones, C. K., Hua, J., Xu, J. & van Zijl, P. C. Imaging of endogenous exchangeable proton signals in the human brain using frequency labeled exchange transfer imaging. Magn. Reson. Med. 10.1002/mrm.24655 (2013).
62. Lin, C. Y., Yadav, N. N., Friedman, J. I., Ratnakar, J., Sherry, A. D. & van Zijl, P. C. Using frequency-labeled exchange transfer to separate out conventional magnetization transfer effects from exchange transfer effects when detecting paracest agents. Magn. Reson. Med. 67, 906-911 (2012).
63. Friedman, J. I., McMahon, M. T., Stivers, J. T. & Van Zijl, P. C. M. Indirect detection of labile solute proton spectra via the water signal using frequency-labeled exchange (flex) transfer. J. Am. Chem. Soc. 132, 1813-1815 (2010).
64. Song, X., Gilad, A. A., Joel, S., Liu, G., Bar-Shir, A., Liang, Y., Gorelik, M., Pekar, J. J., van Zijl, P. C., Bulte, J. W. & McMahon, M. T. Cest phase mapping using a length and offset varied saturation (lovars) scheme. Magn. Reson. Med. 10.1002/mrm.23312 (2012).

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A method of planning, guiding and/or monitoring a therapeutic procedure, comprising:
receiving a non-labeled therapeutic agent by a subject, said non-labeled therapeutic agent comprising at least one type of water-exchangeable proton that is exchangeable with protons in surrounding water molecules so as to enhance detection by a chemical exchange saturation transfer (CEST) process;
acquiring a plurality of CEST magnetic resonance images of said non-labeled therapeutic agent within a region of interest of said subject for a corresponding plurality of times; and
assessing at least one of a therapeutic plan or therapeutic effect of said non-labeled therapeutic agent in tissue of said subject based on said plurality of magnetic resonance images.

2. The method according to claim 1, wherein:
the CEST magnetic resonance images indicate a spatial distribution of the non-labeled therapeutic agent in the tissue, and
the at least one of a therapeutic plan or therapeutic effect is based on an assessment of the spatial distribution of the non-labeled therapeutic agent in the tissue.

3. The method according to claim 2, wherein:
the CEST magnetic resonance images indicate an effective concentration of the non-labeled therapeutic agent in the tissue, and
the at least one of a therapeutic plan or therapeutic effect is based on an assessment of the effective concentration.

4. The method according to claim 1, further comprising:
acquiring a plurality of CEST magnetic resonance images of enzymes linked to the non-labeled therapeutic agent;
assessing activity of the enzymes based on said plurality of magnetic resonance images; and
predicting resistance to the non-labeled therapeutic agent based on the activity of the enzymes.

5. The method according to claim 1, wherein said water exchangeable proton corresponds to at least one of an OH, NH2 or NH group.

6. The method according to claim 1, further comprising determining a pH of the region of interest based on a CEST signal of the CEST magnetic resonance images of the non-labeled therapeutic agent.

7. The method according to claim 6, wherein the CEST signal indicates penetration of the therapeutic agent in the region of interest.

8. The method according to claim 6, wherein:
the non-labeled therapeutic agent includes at least two different types of water exchangeable protons, and
the determining a pH is based on a ratio of CEST signals from the two different types of water exchangeable protons.

9. The method according to claim 1, wherein said non-labeled therapeutic agent is at least one of a drug, a drug analog, or a drug modulator.

10. The method according to claim 1, wherein said non-labeled therapeutic agent is an anticancer drug.

11. The method according to claim 1, wherein said non-labeled therapeutic agent comprises a drug delivery system.

12. The method according to claim 11, wherein said drug delivery system is a nanoparticle drug delivery system.

13. A non-transitory, computer-readable storage medium for planning, guiding and/or monitoring a therapeutic procedure, the computer-readable storage medium comprising computer-executable instructions that, when executed by a computer, cause the computer to perform:
acquiring a plurality of chemical exchange saturation transfer (CEST) magnetic resonance images of a non-labeled therapeutic agent that has been received by a subject, wherein the acquiring step acquires the plurality of magnetic resonance images within a region of interest of said subject for a corresponding plurality of times, said non-labeled therapeutic agent comprising at least one type of water-exchangeable proton that is exchangeable with protons in surrounding water molecules so as to enhance detection by a CEST process; and
assessing at least one of a therapeutic plan or therapeutic effect of said non-labeled therapeutic agent in tissue of said subject based on said plurality of magnetic resonance images.

14. The computer-readable storage medium according to claim 13, wherein:
the CEST magnetic resonance images indicate a spatial distribution of the non-labeled therapeutic agent in the tissue, and
the at least one of a therapeutic plan or therapeutic effect is based on an assessment of the spatial distribution of the non-labeled therapeutic agent in the tissue.

15. The computer-readable storage medium according to claim 14, wherein:
the CEST magnetic resonance images indicate an effective concentration of the non-labeled therapeutic agent in the tissue, and
the at least one of a therapeutic plan or therapeutic effect is based on an assessment of the effective concentration of the non-labeled therapeutic agent in the tissue.

16. The computer-readable storage medium according to claim 13, further comprising computer-executable instructions that, when executed by a computer, cause the computer to perform:
acquiring a plurality of CEST magnetic resonance images of enzymes linked to the non-labeled therapeutic agent;
assessing activity of the enzymes based on said plurality of magnetic resonance images; and
predicting resistance to the non-labeled therapeutic agent based on the activity of the enzymes.

17. The computer-readable storage medium according to claim 13, wherein said water exchangeable proton corresponds to at least one of an OH, NH2 or NH group.

18. The computer-readable storage medium according to claim 17, further comprising instructions that, when executed by a computer, cause the computer to perform:
determining a pH of the region of interest based on a CEST signal of the CEST magnetic resonance images of the non-labeled therapeutic agent.

19. The computer-readable storage medium according to claim 18, wherein the CEST signal indicates penetration of the therapeutic agent in the region of interest.

20. The computer-readable storage medium according to claim 18, wherein:
the non-labeled therapeutic agent includes at least two different types of water exchangeable protons, and
the determining a pH is based on a ratio of CEST signals from the two different types of water exchangeable protons.

21. The computer-readable storage medium according to claim 13, wherein said non-labeled therapeutic agent is at least one of a drug, a drug analog, or a drug modulator.

22. The computer-readable storage medium according to claim 13, wherein said non-labeled therapeutic agent is an anticancer drug.

23. The computer-readable storage medium according to claim 13 wherein said non-labeled therapeutic agent comprises a drug delivery system.

24. The computer-readable storage medium according to claim 23, wherein said drug delivery system is a nanoparticle drug delivery system.

25. A system for planning, guiding and/or monitoring a therapeutic procedure, comprising:
a data processing system; and
a display system configured to communicate with said data processing system,
wherein said data processing system comprises non-transitory, computer-executable instructions that, when executed by said data processing system, causes the data processing system to perform:
acquiring a plurality of chemical exchange saturation transfer (CEST) magnetic resonance images of a non-labeled therapeutic agent that has been received by a subject, wherein the acquiring step acquires the plurality of magnetic resonance images within a region of interest of said subject for a corresponding plurality of times, said non-labeled therapeutic agent comprising at least one type of water-exchangeable proton that is exchangeable with protons in surrounding water molecules so as to enhance detection by a CEST process; and
assessing at least one of a therapeutic plan or therapeutic effect of said non-labeled therapeutic agent in tissue of said subject based on said plurality of magnetic resonance images.

26. The system according to claim 25, wherein:
the CEST magnetic resonance images indicate a spatial distribution of the non-labeled therapeutic agent in the tissue, and
the at least one of a therapeutic plan or therapeutic effect is based on an assessment of the spatial distribution of the non-labeled therapeutic agent in the tissue.

27. The system according to claim 26, wherein:
the CEST magnetic resonance images indicate an effective concentration of the non-labeled therapeutic agent in the tissue, and
the at least one of a therapeutic plan or therapeutic effect is based on an assessment of the effective concentration of the non-labeled therapeutic agent in the tissue.

28. The system according to claim 25, wherein said data processing system comprises non-transitory, computer-executable instructions that, when executed by said data processing system, causes the data processing system to perform:
acquiring a plurality of CEST magnetic resonance images of enzymes linked to the non-labeled therapeutic agent;
assessing activity of the enzymes based on said plurality of magnetic resonance images; and
predicting resistance to the non-labeled therapeutic agent based on the activity of the enzymes.

29. The system according to claim 25, wherein said water exchangeable proton corresponds to at least one of an OH, NH2 or NH group.

30. The system according to claim 29, wherein said data processing system comprises non-transitory, computer-executable instructions that, when executed by said data processing system, causes the data processing system to perform:
determining a pH of the region of interest based on a CEST signal of the CEST magnetic resonance images of the non-labeled therapeutic agent.

31. The system according to claim 30, wherein the CEST signal indicates penetration of the therapeutic agent in the region of interest.

32. The system according to claim 30, wherein:
the non-labeled therapeutic agent includes at least two different types of water exchangeable protons, and
the determining a pH is based on a ratio of CEST signals from the two different types of water exchangeable protons.

33. The system according to claim 25, wherein said non-labeled therapeutic agent is at least one of a drug, a drug analog, or a drug modulator.

34. The system according to claim 25, wherein said non-labeled therapeutic agent is an anticancer drug.

35. The system according to claim 25, wherein said non-labeled therapeutic agent comprises a drug delivery system.

36. The system according to claim 35, wherein said drug delivery system is a nanoparticle drug delivery system.
